(19) United States
(12) Patent Application Publication
(10) Pub. No.: US 2013/0116631 A1
(43) Pub. Date: May 9, 2013

(54) SAFETY NEEDLE ASSEMBLY WITH CORRECT MEDICATION CONNECTION

(71) Applicants: Lynn A. Ziman, Swanzey, NH (US); Steven Huu Nguyen, North Brunswick, NJ (US)

(72) Inventors: Lynn A. Ziman, Swanzey, NH (US); Steven Huu Nguyen, North Brunswick, NJ (US)

(73) Assignee: SMITHS MEDICAL ASD, INC., Rockland, MA (US)

(21) Appl. No.: 13/714,501

(22) Filed: Dec. 14, 2012

Related U.S. Application Data
(63) Continuation of application No. 11/342,620, filed on Jan. 31, 2006.

(51) Int. Cl. A61M 5/32

(52) U.S. Cl. CPC A61M 5/3293
USPC 604/257; 604/272; 604/533

(57) ABSTRACT

An adapter is placed between the needle and a conventional fluid store, or medical line. The end of the adapter that connects to the needle assembly is formed to have a configuration that is complementary to that of the hub of the needle assembly, so that the adapter and the needle hub may be readily mated to each other. The other end of the adapter is formed to have another configuration, for example a conventional luer, that is readily connectable to a conventional fluid store, such as a syringe that has a conventional luer. The adapter is formed by inter-fitting two elements to effect an integral locking mechanism to prevent the uncoupling of the fluid store and the adapter, once they are securely coupled.

This application is a continuation of application No. 11/342,620 filed on Jan. 31, 2006.

### FIELD OF THE INVENTION

The present invention relates to a needle assembly, such as for example a spinal or epidural needle assembly, and more particularly to a needle assembly that is designed to mate with an adapter for correct connection to a particular medication store or line.

### BACKGROUND OF THE INVENTION

To prevent mis-connection of a fluid line or a fluid store to a needle, the prior art teaches the use of a two-part connector with complementary configured opposing surfaces. Such two-part connector ensures that one line from one portion of the connector would not be wrongly connected to a different medication line, and is disclosed in U.S. Pat. No. 6,612,624 and its parent U.S. Pat. No. 6,402,207. The disclosure of the '624 patent is incorporated by reference herein.

An adapter that may be used in place of the connector disclosed in the '624 patent is disclosed in U.S. application Ser. No. 10/915,574 filed on Aug. 11, 2004, assigned to same assignee as the instant application. The disclosure of the '574 application is incorporated by reference herein.

As the connector of the '624 patent requires that there be a two-part connection, in order to use a needle, there is the requirement that a fluid line that converts the input of the needle be added. This is an inconvenience, not to mention the consumption of valuable time, in a medical environment where potentially every second counts.

### SUMMARY OF THE PRESENT INVENTION

The instant invention needle assembly includes a needle that has a needle hub specifically configured to have formation(s) thereon that allows it to be connected only to one end of an adapter, which is configured to have a complementary configuration that allows it and the needle hub to readily mate with each other. The other end of the adapter has a conventional receptacle end, which may be in the form of a luer that allows it to be connected to a conventional luer fitted medication store, such as for example a syringe or a medication fluid line. The hub of the needle assembly of the instant invention, as it is configured to have a particular configuration, is not fittable to a conventional luer. Accordingly, the needle assembly could not be mistakenly connected to a fluid store that may contain medicament that, if injected to a patient, may cause harm to the patient.

As the needle may need to be removed from the fluid store, and/or additional medication be provided to the patient, to ensure that the proper medicament is provided to the patient, the adapter that connects the needle to the appropriate medicament store (or fluid line) has a lock mechanism that prevents the removal of the fluid store once the correct fluid store is connected to the adapter. This ensures that no more medication than necessary be injected to the patient, and also that the correct medicament be provided to the patient from the correct mating of the medicament store and the needle.

The lock mechanism for the instant invention is an integral part of the adapter in that the adapter is made up of two components, namely an adapter core and a shroud that fits about the adapter core. The adapter core is fitted to the shroud during manufacturing. The adapter core has a pair of pawls formed at a substantially central portion of its circumferential outer surface or wall. These pawls act against ramped stops formed on the interior circumferential surface or wall of the shroud, when the adapter core and the shroud are rotated relative to each other. Once coupled to the adapter, the fluid store (or fluid line) may no longer be removed from the adapter, as rotation in the direction that ordinarily would have uncoupled the receptacle end of the fluid store from the adapter would cause the receptacle end of the adapter to rotate in unison with the fluid store, thereby preventing the receptacle end of the fluid store and the receptacle end of the adapter from disengaging. As a result, whatever medicament stored in the fluid store that is meant to be used with the needle, which would only mate with the particular adapter, could only be used with the needle, thereby preventing any possible mis-connection of a different medicament container to the needle, which may still be inserted to the patient.

The instant invention therefore relates to an apparatus that comprises a needle assembly that includes a hub having a given configuration at its receptacle end and a needle extending from its closed end, and an adapter having a first end with a first configuration complementary to the given configuration for mating with the hub at its receptacle end. The adapter further has a second end with a second configuration so that a fluid store, or a fluid line, that has a receptacle end with a configuration complementary to the second configuration is adaptable to mate with the second end of the adapter.

The instant invention also relates to a combination in which a needle assembly having a needle hub with a receptacle end of a given configuration and a needle extending from its closed end is combined with an adapter having a first end with a configuration complementary to the given configuration so that the needle hub and the first end of the adapter are readily matable with each other. The adapter has a second end with a second configuration that allows the adapter to be coupled to the receptacle end of a fluid store, or a fluid line. The adapter further acts as a locking mechanism to prevent the fluid store (or the fluid line), once coupled to the adapter, from rotating in a direction relative to the adapter that allows the fluid store from being uncoupled from the adapter.

The instant invention further relates to a method of coupling a needle assembly including a hub having a receptacle end and a needle extending from its closed end to an appropriate medicament fluid store or fluid line. The method includes the steps of: a) effecting a given configuration at the receptacle end of the hub; b) providing an adapter with a first end having a first configuration complementary to the given configuration for mating with the hub at its receptacle end; c) effecting a second configuration to a second send of the adapter; and d) providing a fluid store or a fluid line having a receptacle end with a configuration complementary to the second configuration for mating with the second end of the adapter.

### BRIEF DESCRIPTION OF THE FIGURES

The present invention will become apparent and the invention itself will be best understood with reference to the following description of the present invention taken in conjunction with the accompanying drawings, wherein:

FIG. 1 illustrates a needle of the instant invention with an adapter and a fluid store;

FIG. 2 is a view illustrating the disassembled components of the adapter, as it relates to the needle;

FIGS. 3a and 3b are side views of the needle assembly of the instant invention;

FIGS. 4a-4f are different views of the adapter core component of the adapter;

FIGS. 5a-5g are different views of the shroud component of the adapter; and

FIG. 6 shows a filter interposed between the adapter and the needle assembly.

### DETAILED DESCRIPTION OF THE INVENTION

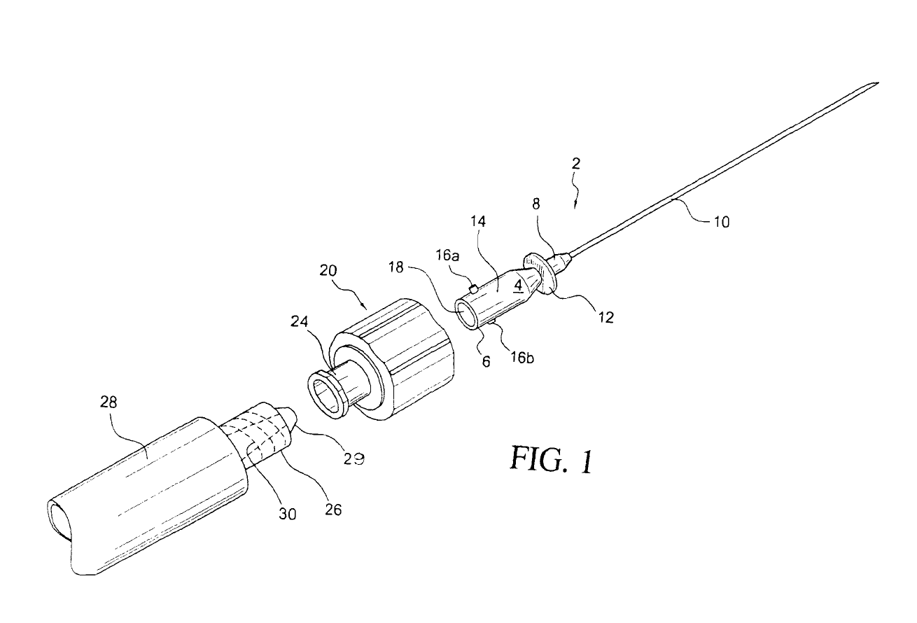

The instant invention, as shown in FIG. 1, includes a needle assembly 2 that has a needle hub 4. Needle hub 4 has a receptacle end 6 and a closed end 8 from which a needle 10 extends. Needle 10 may be a conventional needle, but for the instant embodiment is an epidural or a spinal needle for insertion to a patient. As shown, the closed end 8 is separated from the rest of needle hub 4 by a flange 12.

Receptacle end of needle hub 4 comprises an elongate cylindrical portion whereat a particular formation, or formations, are effected during the manufacturing process for providing a particular or given configuration that is unique to the needle assembly. For the embodiment shown, there are two protrusions 16a and 16b formed at opposite sides of the receptacle end 6. These protrusions, along with the cross sectional dimension of receptacle end 6, provide the given configuration for needle hub 4 of the needle assembly 2. The thickness of the circumferential wall of the elongate cylindrical portion 14 is dimensioned to provide a further attribute of the configuration of needle hub 4, i.e., configuring the cross section of opening 18 at receptacle end 6 and the through passage into needle hub 4. Opening 18 is of a sufficient dimension to accept the receptacle end of an adapter 20 to which needle hub 4 of the needle assembly 2 mates with. Protrusions 16a and 16b are formed on the receptacle end 6 of needle hub 4 a particular distance from opening 18 as part of the formation for effecting the proper mating with adapter 20, more specifically with the receptacle end thereof and the shroud component of adapter 20, to be discussed later.

As further shown in FIG. 1, adapter 20 has another receptacle end 24, which may be configured as a conventional luer end for mating with the conventional luer end 26 of a fluid store, such as for example a syringe 28. In place of a fluid store, a fluid line having fitted to its end a luer such as 26 is also contemplated. Although the receptacle end 26 of syringe 28 is able to mate with receptacle end 24 of a adapter 20, it could not mate with the receptacle end 6 of needle assembly 2. Receptacle end 26 of syringe 28 has a male receptacle end 29, and is internally threaded, as represented by the dotted threaded line 30.

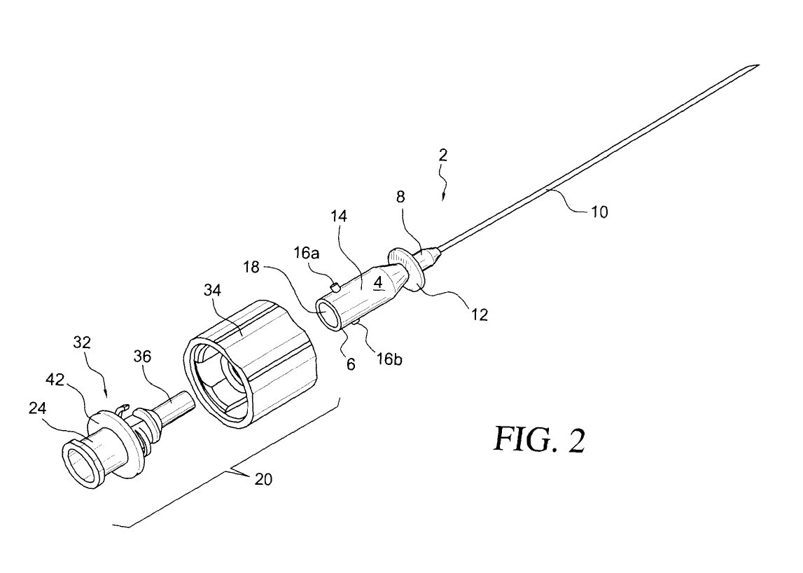

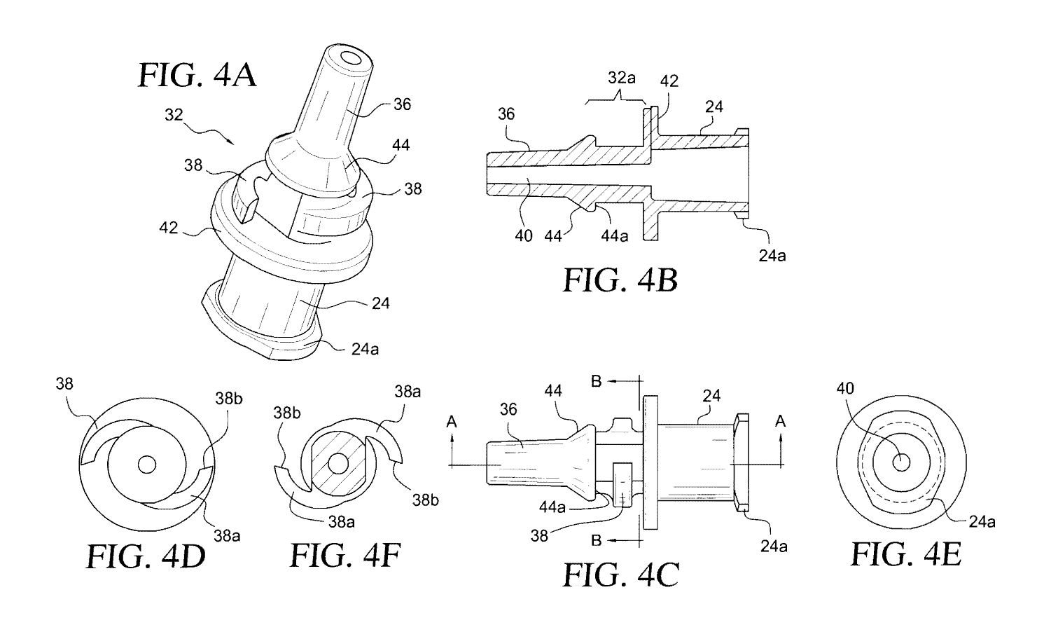

FIG. 2 shows the different components of adapter 20 in relation to needle assembly 2. As shown, adapter 20 comprises an adapter core 32 and a shroud 34 that is adapted to fit about core 32. As mentioned previously, adapter core 32 has a receptacle end 24, which may also be referred to as its second or distal end. In addition, core 32 has a first receptacle end 36, or a first or proximal end, for mating with receptacle end 6 of needle hub 4. The first end 36 and the second end 24 of adapter core 32 are connected, as adapter core 32 is a single molded piece, by a through passage 40 (FIG. 4b) that extends from the opening at first end 36 to the opening at second end 24. Shroud 34 and adapter core 32 are separate pieces that may be molded from conventional medical plastics.

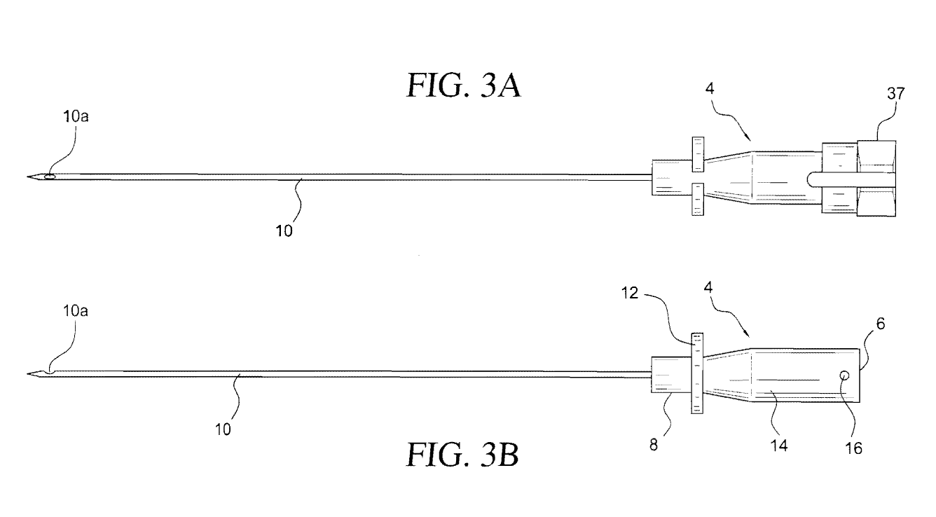

Needle assembly is shown in side views 3a and 3b. FIG. 3a shows the receptacle end 6 of needle hub 4 of the needle assembly 2 being covered by a cap 38 that has extending therefrom a thin cannula (not shown) inserted within needle 10 to prevent coring, or the blocking of the needle opening 10a, when needle 10 is inserted to the patient. It is only after needle 10 has been properly inserted into the patient would the user remove cap 38, and therefore the cannula inserted in needle 10. At which time, fluid may pass between needle 10 and the patient through opening 10a at the tip of needle 10.

Adapter core 32 of adapter 20 of the instant invention, as best shown in FIGS. 4a-4f, has an elongate conical first end 36 and a cylindrical second end 24, which has at its end flanges 24a for forming a conventional luer connection. As best shown in FIGS. 4a, 4d and 4f, at the proximate mid-section of adapter core 32 there are two integral pawls 38 formed at the outer circumferential surface or wall of core 32. These pawls each have a semi-ramped portion 38a and an end stop 38b, as best shown in FIG. 4f. As best shown in FIG. 4b, a through passage 40 extends from the opening at first end 36 to the opening at second end 24. A flange 42 that separates the first end from the second end provides a back stop, when adapter core 36 is fitted into shroud 34 (or conversely shroud 34 being fitted about adapter core 32), as shown in FIG. 1. A tapered circumferential portion 44 enables adapter core 32 to be inserted to shroud 34, and be fixedly but rotatably retained within shroud 34.

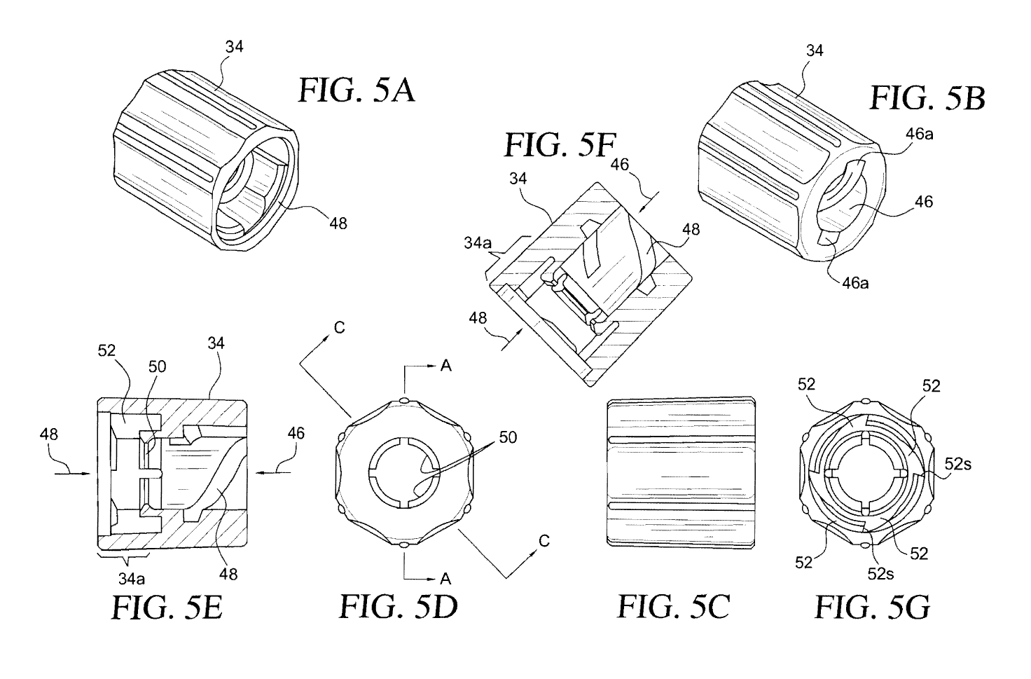

Shroud 34 is shown in FIGS. 5a-5g to have a first opening 46 and a second opening 48. Opening 46 is fabricated to have a configuration that is complementary to the configuration of the formation at receptacle end 6 of needle assembly 2, as discussed above. In particular, as shown in FIG. 5b, opening 46 of shroud 34 has a formation in the shape of a circle, but with two side inlets or channels 46a that allow the corresponding protrusions 16 of needle hub 4 to pass through. As the shroud is formed with an internal thread 48, receptacle end 6 of needle hub 4, once inserted to opening 46, may be secured to shroud 34 by the relative rotation of hub 4 and shroud 34, so that protrusions 16 of needle hub may travel along the internal thread of shroud 34, and receptacle end 6 via its opening 18 be more fully mated with the receptacle end 36 of adapter core 32. Once fully threaded, needle hub 4 is securely mated to adapter 20.

Adapter core 32 is pressedly fitted to shroud 32 in the direction as shown by directional arrow 48 in FIGS. 5e and 5f. The portion of adapter core 32, designated 32a in FIG. 4b, is fitted and held by shroud 34 at portion 34a, as shown in FIGS. 5e and 5f. A number of extensions 50 circumferentially spaced at the interior wall of shroud 34, due to their inherent plastics elasticity, would allow portion 44 of adapter core 32 to pass through, as the latter's front is tapered, but would prevent the same from coming back out of shroud 34, as the circular flat backside 44a of portion 44 is biasedly held by extensions 50. As a result, once adapter core 32 is fitted into shroud 34, it cannot be removed therefrom.

Also provided in shroud 34 are a number of ramped stops 52, as best shown in FIG. 5g, that prevent adapter core 32 from rotating in a direction where the stop surfaces 52s would bias against pawl stops 38b. In practice, if a user were to hold adapter 34 by its receptacle end 24 while attempting to rotate shroud 34, she could only rotate shroud 34 in a counterclockwise direction. Any attempt to rotate shroud 34 relative to adapter core 32 in the clockwise direction would also cause adapter 32 to rotate in unison with shroud 34.

In operation, by rotating luer end 24 of adapter 20 relative to the luer end of a fluid store, such as for example syringe 28 shown in FIG. 1, in a clockwise direction, adapter 20 can readily be coupled to the fluid store. However, any attempt to remove adapter 20 from syringe 28, by for example rotating adapter 2 (more specifically shroud 34) counterclockwise relative to syringe 28 would fail, as adapter core 32 would move (rotation or non-rotation) in unison with syringe 28, while the movement of shroud 34 (non-rotation or rotation) would be independent of the unified movement of adapter core 32 and syringe 28. As a result, once syringe 28 is coupled to adapter 20, it remains coupled thereto, thereby eliminating the real possibility that a different syringe may be mis-connected to needle assembly 2, which hub has a configuration that could only mate with the complementary configuration at receptacle end 36 of adapter 20.

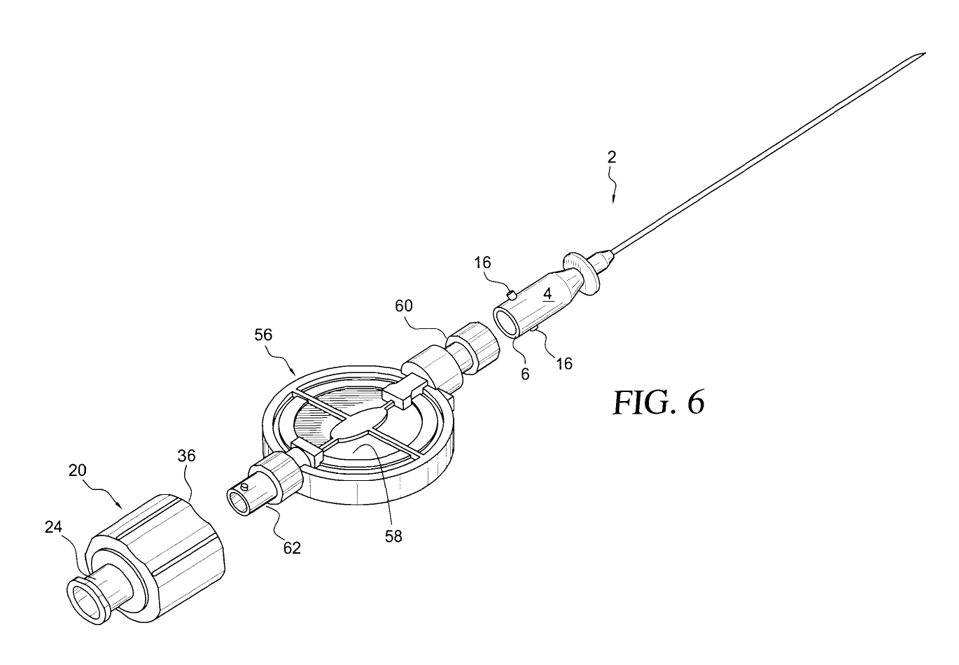

Another aspect of the instant invention is shown in FIG. 6. There a filter device 56 is shown to be interposedly connectable to adapter 20 and needle assembly 2. Filter device 56, in addition to having a filter element 58, has a first end 60 that has a configuration that is complementary to the configuration of the receptacle end 6 of needle hub 4 of the needle assembly 2. In other words, the configuration of the receptacle end 60 for filter device 56 is the same as the configuration of the receptacle end 36 of adapter 20. Further, filter device 56 has a second end 62 for mating with adapter 20 that has a configuration that is the same as the configuration of receptacle end 6 of needle hub 4. Once fully connected to both needle assembly 2 and adapter 20, filter device 56 filters the fluid passing between the needle assembly 2 and adapter 20, and of course the fluid store that is connected to end 24 of adapter 20. The filter element 58 of filter device 56 may be any conventional medical filter that is adaptable to filter out undesirable particles or elements that may be in the fluid.

1. A needle hub comprising a one piece elongate cylindrical portion with an open end having an opening defined by a circumferential wall and a closed end, the open end forming a hub receptacle end having a particular formation that prevents the hub receptacle end from mating with a conventional luer end of a medical device, said particular formation including two protrusions formed at opposite sides of the hub receptacle end at a particular distance from the opening of the open end so that the protrusions are threadedly matable with an internal thread formed at an inner circumferential wall of a shroud having a complementary configuration to said particular formation, the hub receptacle end with the protrusions not matable with an internally threaded conventional luer end, said particular formation further including the circumferential wall of the elongate cylindrical portion being dimensioned to provide a given cross section for the opening at the hub receptacle end that prevents a conventional male receptacle end from being fittingly accepted into the opening.

2. Needle hub of claim 1, further having a needle extending from the closed end.

3. Needle hub of claim 1, wherein the shroud fits about a non-conventional receptacle end of a core, and wherein the non-conventional receptacle end is fittingly accepted into the opening of the hub receptacle end when the two protrusions are threadedly mated with the internal thread formed at the inner circumferential wall of the shroud.

4. Needle hub of claim 3, wherein the shroud and the core are adapted to be a non-removable connector end of a fluid store.

5. Needle hub of claim 3, wherein the hub receptacle end and the non-conventional receptacle end are removable from each other when the shroud and the elongate cylindrical portion are rotated relative to each other in a direction counter to the direction that threadedly mates the protrusions of the hub receptacle end with the internal thread of the shroud.

6. Needle hub of claim 2, wherein the needle is an epidural or spinal needle.

7. Needle hub of claim 2, further comprising a flange at the closed end separating the needle from the rest of the needle hub.

8. A one piece needle hub comprising with an open end having an opening defined by a circumferential wall and a closed end having a needle extending therefrom, the open end forming a hub receptacle end having a particular formation that prevents the hub receptacle end from mating with a conventional luer end of a medical device, said particular formation including two protrusions formed at opposite sides of the hub receptacle end at a particular distance from the opening of the open end so that the protrusions are threadedly matable with an internal thread formed at an inner circumferential wall of a shroud having a complementary configuration to said particular formation, the hub receptacle end with the protrusions not matable with an internally threaded conventional luer end, said particular formation further including the circumferential wall of the hub receptacle end defining the opening to have a dimension that prevents the opening at the hub receptacle end from fittingly accepting a conventional male receptacle end.

9. Needle hub of claim 8, wherein the shroud fits about a non-conventional receptacle end of a core, and wherein the non-conventional receptacle end is fittingly accepted into the opening of the hub receptacle end when the two protrusions are threadedly mated with the internal thread formed at the inner circumferential wall of the shroud.

10. Needle hub of claim 8, wherein the shroud and the core are adapted to be a non-removable connector end of a fluid store.

11. Needle hub of claim 8, wherein the hub receptacle end is removable from the non-conventional receptacle end and the shroud by rotating the elongate cylindrical portion and the shroud relative to each other in a direction counter to the direction that threadedly mates the protrusions of the hub receptacle end with the internal thread of the shroud.

12. Needle hub of claim 8, wherein the needle is an epidural or spinal needle.

13. Needle hub of claim 8, further comprising a flange at the closed end separating the needle from the rest of the needle hub.

14. A system comprising:
a one piece needle hub with an open end having an opening defined by a circumferential wall and a closed end having a needle extending therefrom, the open end forming a hub receptacle end having a particular formation that prevents the hub receptacle end from mating with a conventional luer end of a medical device, said particular formation including two protrusions formed at opposite sides of the hub receptacle end at a particular distance from the opening at the open end and the opening of the hub receptacle end dimensioned to have a cross section that prevents the opening from fittingly accepting a conventional male receptacle end; and
a fluid store adapted to have a non-removable connector end having a complementary configuration to said particular formation, the connector end including a shroud that has an internal thread formed at its inner circumferential wall that is threadedly matable with the protrusions at the hub receptacle end, the complementary configuration of the connector end preventing the connector end from being connected to a conventional receptacle end of a conventional needle hub.

15. System of claim 14, wherein the shroud fits about a non-conventional receptacle end of a core at the connector end, and wherein the non-conventional receptacle end is fittingly accepted into the opening of the hub receptacle end when the two protrusions at the hub receptacle end are threadedly mated with the internal thread at the inner circumferential wall of the shroud.

16. System of claim 15, wherein the core comprises a through passage to the fluid store.

17. System of claim 14, wherein the hub receptacle end and the non-conventional receptacle end are removable from each other when the needle hub and the shroud are rotated relative to each other in a direction counter to the direction that threadedly mates the protrusions at the hub receptacle end with the internal thread of the shroud.

18. System of claim 14, wherein the fluid store and the connector end are separate components with respective counterpart receptacle ends that are matable to each other, the fluid store and the connector end not removable from each other once fully mated to each other.

19. System of claim 14, wherein the needle is an epidural needle.

20. System of claim 14, further comprising a flange at the closed end separating the needle from the rest of the needle hub.
